(19) United States
(12) Patent Application Publication
(10) Pub. No.: US 2025/0132032 A1
(43) Pub. Date: Apr. 24, 2025

(54) SYSTEMS AND METHODS FOR PROVIDING EXTENDED REALITY PATIENT EDUCATION

(71) Applicant: HouseCall VR Inc., Media, PA (US)

(72) Inventors: Linda Heer Ciavarelli, Media, PA (US); Anthony Gennaro Ciavarelli, III, Media, PA (US)

(73) Assignee: HouseCall VR Inc., Media, PA (US)

(21) Appl. No.: 18/918,744

(22) Filed: Oct. 17, 2024

Related U.S. Application Data
(60) Provisional application No. 63/591,239, filed on Oct. 18, 2023.

(51) Int. Cl. G16H 40/67; G06F 3/01; G16H 15/00; G16H 50/20

(52) U.S. Cl. CPC G16H 40/67; G06F 3/011; G16H 15/00; G16H 50/20

(57) ABSTRACT

A method of delivering interactive content includes storing content elements in an elements library. The method includes receiving a request for a content module. The request includes an identification of a subject of the content module. Additionally, the method includes extracting, from the elements library, one or more extended reality content elements based on the subject of the content module. Further, the method includes generating the content module from on the one or more extended reality content elements. The method includes storing the content module in a computer-readable storage medium.

### CROSS-REFERENCE TO RELATED APPLICATIONS

This application claims the benefit of priority of U.S. provisional application No. 63/591,239, filed Oct. 18, 2023, titled “SYSTEMS AND METHODS FOR PROVIDING EXTENDED REALITY PATIENT EDUCATION,” the entire contents of which are herein incorporated by reference.

### FIELD

The present disclosure relates to interactive computer systems and methods and, more particularly, to extended reality computer systems and methods.

### BACKGROUND

Over the course of modern medicine, healthcare professionals have looked for innovative ways to treat their patients through new technologies. Similar technological advancements in patient education have not met this evolution in treatment. In most settings, healthcare professionals rely upon verbal and written explanations to patients. This often leads to patients not understanding what their healthcare professionals are telling them and healthcare professionals not having sufficient time to explain.

As can be seen, there is a need for technological systems and processes to address the above problems.

### SUMMARY

In one aspect of the present disclosure, a method of delivering interactive content includes storing content elements in an elements library. The method includes receiving a request for a content module. The request includes an identification of a subject of the content module. Additionally, the method includes extracting, from the elements library, one or more content elements based on the subject of the content module. Further, the method includes generating the content module from the one or more content elements. The method includes storing the content module in a computer-readable storage medium. The system also includes an extended reality content playback device coupled to the delivery system by one or more networks. The extended reality content playback device is configured to receive the content module. The extended reality content playback device is configured to execute the content module for viewing and interaction by a user.

In another aspect of the present disclosure, a system of delivering interactive content includes a content generation system. The content generation system is configured to store content elements in an elements library. The content generation system is configured to receive a request for a content module. The request includes an identification of a subject of the content module. The content generation system is configured to extract, from the elements library, one or more content elements based on the subject of the content module. The content generation system is configured to generate the content module from the one or more content elements. The content generation system is configured to store the content module in a computer-readable storage medium. The system also includes the delivery system described in the paragraph above. The delivery system is configured to receive content modules from the content generation system by one or more networks.

### BRIEF DESCRIPTION OF THE DRAWINGS

FIG. 1A is a diagram of an interactive healthcare environment, including an extended reality education system, according to aspects of the present disclosure;

FIG. 1B is a diagram of an extended reality education system in the interactive healthcare environment, according to aspects of the present disclosure;

FIG. 2 is a diagram of an extended reality system that can be used in the interactive healthcare environment of FIG. 1A, according to aspects of the present disclosure;

FIG. 3 is a flow diagram of a method of creating and using content modules in the interactive healthcare environment of FIG. 1, according to aspects of the present disclosure;

FIG. 4 is a process diagram for generating content modules using the extended reality education system, according to aspects of the present disclosure;

FIGS. 5A-5C are diagrams of an arrangement of content module, according to aspects of the present disclosure; and

FIG. 6 is a diagram of an arrangement of one section of a content module, according to aspects of the present disclosure.

### DETAILED DESCRIPTION OF THE DISCLOSURE

The following detailed description is of the best currently contemplated modes of carrying out exemplary embodiments of the disclosure. The description is not to be taken in a limiting sense but is made merely for the purpose of illustrating the general principles of the disclosure, since the scope of the disclosure is best defined by the appended claims.

Currently, the options for patient education consist of written materials (i.e., brochures and other written materials) or two-dimensional materials that are delivered via a web portal or video. The current options are not providing educational content in a manner that can be understood and/or retained by most patients. Per a 2022 scoping review article in Patient Education and Counseling, researchers noted that 40-80% of patients could not accurately recall information shared with them within 90 minutes of a visit to their healthcare provider, and 50% of medication information was misunderstood by the patient immediately following the visit.

Traditional patient education methods often rely on static materials and one-size-fits-all approaches. With the advent of extended reality technology, there's an opportunity to create immersive, interactive, and tailored educational experiences for patients, enhancing understanding and retention.

Broadly, an embodiment of the present disclosure provides an extended reality educational (“XR ED”) system and methods for creating and delivering an interactive educational experience. The XR ED system utilizes virtual reality/augmented reality/mixed reality (together, “extended reality” or “XR”) and leverages artificial intelligence (AI) to produce immersive learning experiences for patients across the healthcare spectrum. The XR ED system generates and delivers a user-friendly interface for building patient educational experiences. The XR ED system also generates and delivers an interface for healthcare professional staff to manage and deliver content to patients. The XR ED system also generates and delivers user-friendly interfaces that allows patient to view records and content modules directly. The XR ED system addresses the limitations of existing patient education methods by leveraging XR and AI technology to create immersive educational experiences that will vastly enhance patient understanding and retention as compared to today's patient education delivery methods. Several studies have shown the benefits of utilizing VR for educational training purposes. For example, a study in Surg Endosc. (2018 August) noted that VR-trained students showed a 250% improvement in their ability to accurately complete a safety procedure. The XR ED system translates these types of improvements that VR imparts into educational modules for patients.

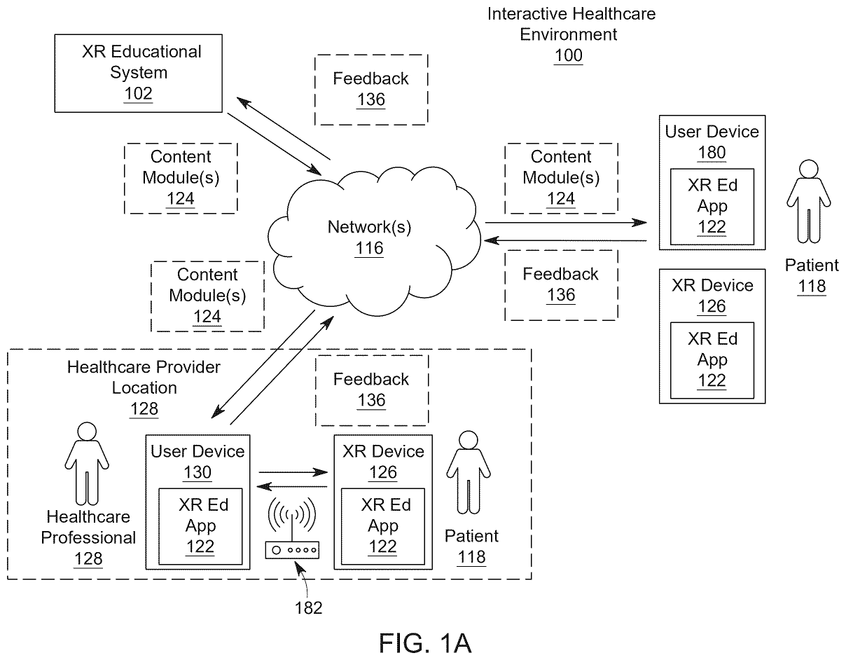

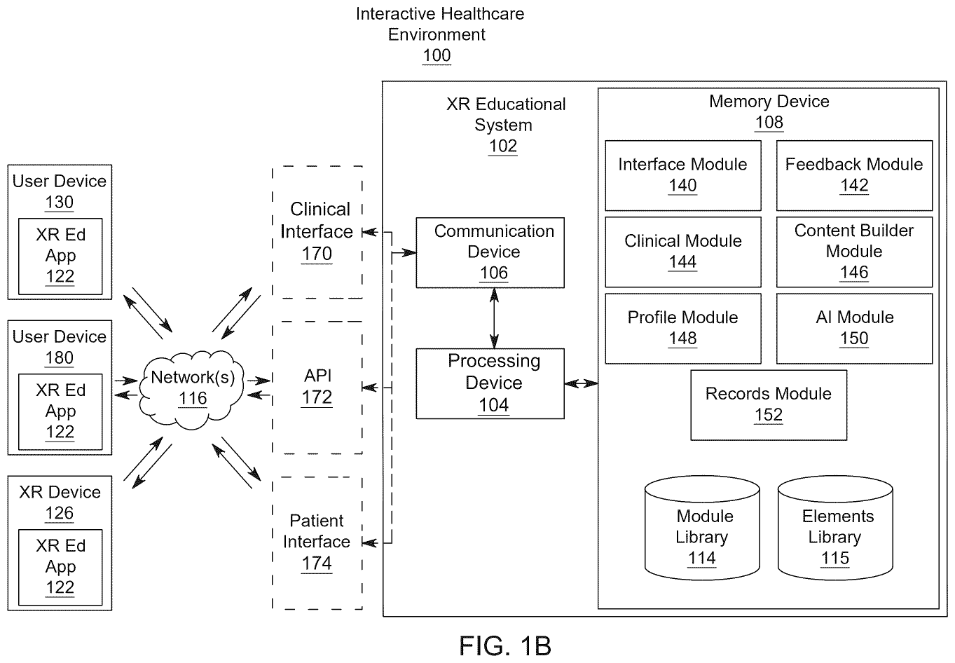

Referring now to FIGS. 1A, 1B, and 2-6, FIGS. 1A and 1B illustrate an interactive healthcare environment 100 including an XR ED system 102, according to aspects of the present disclosure. While FIGS. 1A and 1B illustrate examples of components of the interactive healthcare environment 100 and the XR ED system 102, additional components can be added, and existing components can be removed and/or modified.

The XR ED system 102 revolutionizes health literacy by creating a universal language between patients/customers (defined herein as “patient”) and healthcare professionals (including, but not limited to, doctors, physician's assistants, nurses, pharmacists, and allied health professionals) through extended reality. The XR ED system 102 utilizes immersive XR technologies such as virtual reality (VR), augmented reality (AR), mixed reality (MR), and metaverse-based social platforms to better share health information. Patients will learn through these first-person educational experiences that will be made available across multiple platforms. The XR ED system 102 goes beyond traditional forms of written and verbal patient education to provide healthcare professionals and their patients with immersive, extended reality modules that will more fully explain healthcare conditions. The XR ED system 102 builds these XR educational experiences with patient understanding and healthcare professional efficiency as their focus. The XR ED system 102 can deliver all types of patient education that healthcare professionals describe every day through the system's library of conditions and treatment programs across healthcare disciplines. The XR ED system 102 will provide a one-stop-shop for patient education and healthcare professional efficiency.

As illustrated in FIG. 1A, in the interactive healthcare environment 100, the XR ED system 102 communicates with a healthcare provider location 128 via one or more networks 116. For example, the healthcare provider location 128 can include a user device 130 that is utilized by a healthcare provider 128. The healthcare provider 128 can utilize the user device 130 to communicate with the XR ED system 102 to request content module(s) 124 for playback on one or more XR device(s), for example, an XR device 126. The healthcare provider 128 can utilize the user device 130 to select, configure, and customize the content module(s) 124 for a user, for example, a patient 118, to view on the XR device 126. In embodiments, the user device 130 and the one or more XR device(s) 126 can communicate via a router 182. The router 182 can be configured to establish a secure communication channel between the user device 130 and the one or more XR device(s) 126. Likewise, the router 182 can operate as a secure gateway to the XR education system 102 for the user device 130 and the one or more XR device(s) 126.

In some embodiment, the content module(s) 124 can be directly provided to a user device 180 and/or a XR device 126 via the networks 116, which are controlled by the patient 118. The patient 118 can utilize the user device 180 and XR device 126 to select content modules, review viewing history, view their medical records and the like. While FIG. 1A illustrates one healthcare provider location 128 with one user device 130 and one XR device 126, the interactive health care environment 100 can include any number of the healthcare provider locations 128 and any number of user devices 130 and/or XR devices 126, whether located at the healthcare provider locations 128 or independently.

The XR device 126 can present one or more content module(s) 124 to a user of the XR device 126, for example, the patient 118. The user, e.g., the patient 118, views the content module(s) 124 and interacts with the XR device 126 to provide feedback 136 on the content module(s) 124. The feedback 136 from the user, e.g., the patient 118, can include user input(s), including but not limited to: voice input from patient 118 during use of XR device 126; data gathered from patient 118 in response to quizzes and other “teach-back” components completed during use of XR device 126; and biometric information of patient 118 tracked by the XR device 126, e.g., eye movement, head movement, heart rate, brain waves, etc. The healthcare professional 128 can also provide feedback 136 using the user device 130. In some embodiments, the content module(s) 124 can be designed to provide extended reality content on computing devices, for example, the XR device 126. In some embodiments, the content module(s) 124 can provide content that can be viewed on conventional computer devices such as mobile phones, laptops, desktops, smart appliances, etc.

To select, configure, customize, and/or play the content module(s) 124, the user device 130 can store and execute a copy of an XR Ed application 122. Likewise, the XR device 126 can store and execute a copy of XR Ed application 122. Likewise, the user device 180 can store and execute a copy of XR Ed application 122. In some embodiments, the XR Ed application 122 can be a specifically designed application that operates with XR ED system 102 to perform the processes and methods described herein. In some embodiments, the XR Ed application 122 can be a third-party application, such as a web browser, that communicates with the XR ED system 102 to perform the processes and methods described herein.

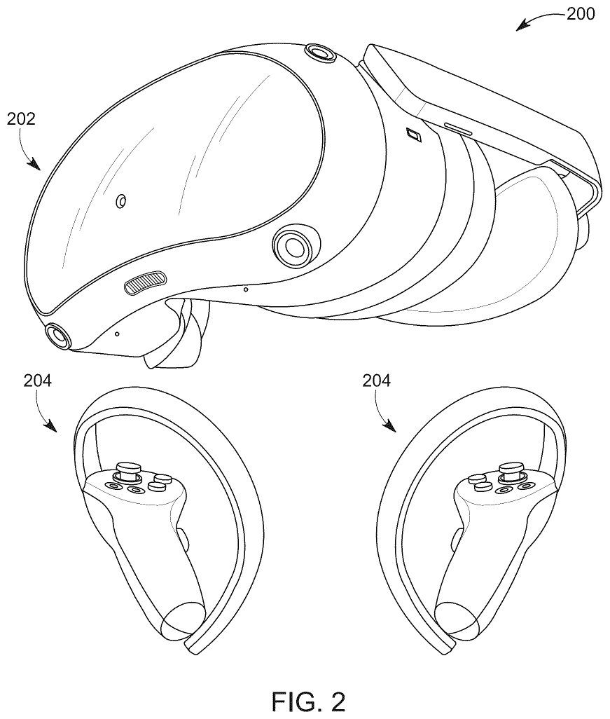

The user device 130 and the user device 180 can include one or more electronic devices such as a laptop computer, a desktop computer, a tablet computer, a smartphone, a thin client, an extended reality system, and the like. FIG. 2 illustrates one example of an XR system or device 200, according to aspects of the present disclosure. In some embodiments, the XR system 200 can be used as the XR device 126 discussed herein. The XR system 200 can include an extended reality headset 202 for delivering visual and audio output to the user and capturing user input and feedback, for example, audio input, hand-tracking, eye movement, facial expression, etc. The XR system 200 can also include one or more controllers 204 for delivering haptic output and receiving input from the user via, for example, buttons, sticks, switches, motion sensors, and the like.

As illustrated in FIG. 1B, the XR ED system 102 includes a processing device 104 coupled to a communication device 106. The processing device 104 is also coupled to a memory device 108. In embodiments, the communication device 106 enables the XR ED system 102 to communicate with other devices and systems via one or more networks 116. The XR ED system 102 can communicate with the user device 130 and/or the XR device 126 via the network 116. For example, the communication device 106 can establish one or more types of interfaces that allow the user device 130, user device 180 and/or the XR device 126 to communicate with the XR ED system 102, for instance, a clinical interface 170 (e.g., a website, an application, a network socket, file transport socket, RSS feed, etc.), a patient interface 174 (e.g., a website, an application, a network socket, file transport socket, RSS feed, etc.) and/or an application programming interface (API) 172.

To perform the process described herein, the XR ED system 102 can store and execute an interface module 140, a feedback module 142, a clinical module 144, a content builder module 146, a profile module 148, an artificial intelligence (AI) module 150, and a records module 152 to perform the processes and methods described herein. The interface module 140, the feedback module 142, the clinical module 144, the content builder module 146, the profile module 148, the AI module 150, and the records module 152 can be stored in the memory device 108. The interface module 140, the feedback module 142, the clinical module 144, the content builder module 146, the profile module 148, the AI module 150, and the records module 152 can include the necessary logic, instructions, and/or programming to perform the processes and methods described herein. The interface module 140, the feedback module 142, the clinical module 144, the content builder module 146, the profile module 148, the AI module 150, and the records module 152 can be written in any programming language.

The memory device 108 can also include one or more databases, for example, a module library 114 and an elements library 115 that stores information and data associated with the process and methods described herein. The module library 114 can store content modules, e.g., content modules 124, that are created by the XR ED system 102 and used in the patient education processes. The elements library 115 can store elements used in the creation of the content modules. The elements can include background music, visual elements, texts, intro, and outro. The visual elements can include categories such as environments, effects, body parts, cellular parts, and miscellaneous others. In some embodiments, the content modules 124 can be two-dimensional modules that can be viewed on user devices such as the user device 130 and/or the user device 180.

The interface module 140 operates to generate and provide graphical user interfaces (GUIs) to the user device 130, the XR device 126, and the user device 180 during the processes. The GUIs generated by the interface module 140 can be interactive. In embodiment, the interface module 140 (or other modules) can support automatic content translation to multiple languages based on patient dialect and preferences and incorporates features for individuals with disabilities, including sign language interpretation and voice command functionalities. Voice command and natural hand gesture functionality allows patients to pose questions or navigate the VR space seamlessly.

The feedback module 142 operates to receive, process, analyze, and store feedback from the users of the XR ED system 102. The feedback can include feedback captured by the user device, e.g., extended reality system 200 and user device 180, feedback provided by a patient, feedback provided by a healthcare provider, and the like. For example, as patients navigate the XR environment, the XR ED system 102 can offer immediate, real-time feedback on their comprehension through quizzes and interactive scenarios, ensuring active engagement and learning reinforcement. Beyond traditional visual and auditory elements, the XR ED system 102 can integrate haptic feedback and potential olfactory stimuli, offering a richer, multi-sensory learning environment. In embodiments, the feedback can be collected and processed after viewing a content module to allow the feedback module 142 to update, customize, and improve the content module(s) 124. In embodiments, the feedback can be collected and processed, in real time, to update the content module(s) 124, in real time, as they are being viewed.

In embodiment, the feedback module 142 can include an integrated tracking system that monitors patient progress over time, highlighting areas of strength and topics requiring further exploration. Advanced sensors and cameras gauge patient emotional responses, allowing the XR ED system 102 to adjust content delivery or pacing. If a patient appears overwhelmed, the software can modify its approach or suggest breaks. Patients can engage in extended reality modules that cover, but are not limited to, the following types of healthcare topics: virtual simulations of medical procedures that patient will undergo; disease and healthcare condition impact and management; medication usage and side effects; or other healthcare scenarios that assist with demystifying processes for patients and alleviating potential patient anxieties and furthering patient understanding of their healthcare conditions.

In embodiment, hand tracking allows patients to interact with the XR environment using natural hand movements. This can be especially useful for simulations where the patient might need to “touch”, “grab” or interact with virtual objects. Recognizing the potential for XR-induced discomfort, the XR ED system 102 incorporates quick exit features and virtual assistants, ensuring patient safety and comfort. Eye tracking monitors where the patient is looking in the VR environment. This can provide insights into which parts of the content are most engaging or confusing. It can also be used to navigate menus or select options just by looking at them. In embodiment, the XR ED system 102 can integrate game-like elements, such as point systems and challenges, to motivate patients and foster competitive learning.

In embodiment, the feedback module 142 can record XR ED system 102 user's responses during each content module 124 using multiple input methods, including but not limited to, voice, eye gaze, or touch, in the framework of a ‘teach-back’ method. These recorded responses are then transcribed and analyzed using artificial intelligence, e.g., one or more trained machine learning models, contained in AI module 150 according to a predefined rubric. For example, AI module 150 can analyze patient 118's responses and inputs after viewing content module 124 to generate a score based on the rubric, which is displayed within patient 118's profile, providing healthcare professional 128 with insight into patient 118's level of understanding of content module 124. This process not only allows for better tracking of patient comprehension but also supports the XR educational system 102 development team by identifying effective content and highlighting areas for improvement. Additionally, the XR educational system 102 generates useful data on user performance and engagement for further analysis and other uses.

The clinical module 144 operates to provide an interface portal for healthcare providers to interact with the XR ED system 102, for example, through user device 130 and clinical interface 170 The clinical module 144 can be configured to provide a login interface that establishes a secure session for the healthcare provider to select content modules and direct the content modules to the user device of the patient, e.g., XR device 126 and user device 180. The clinical module 144 can also provide an interface portal for the patients 118 to interact with the XR ED system 102.

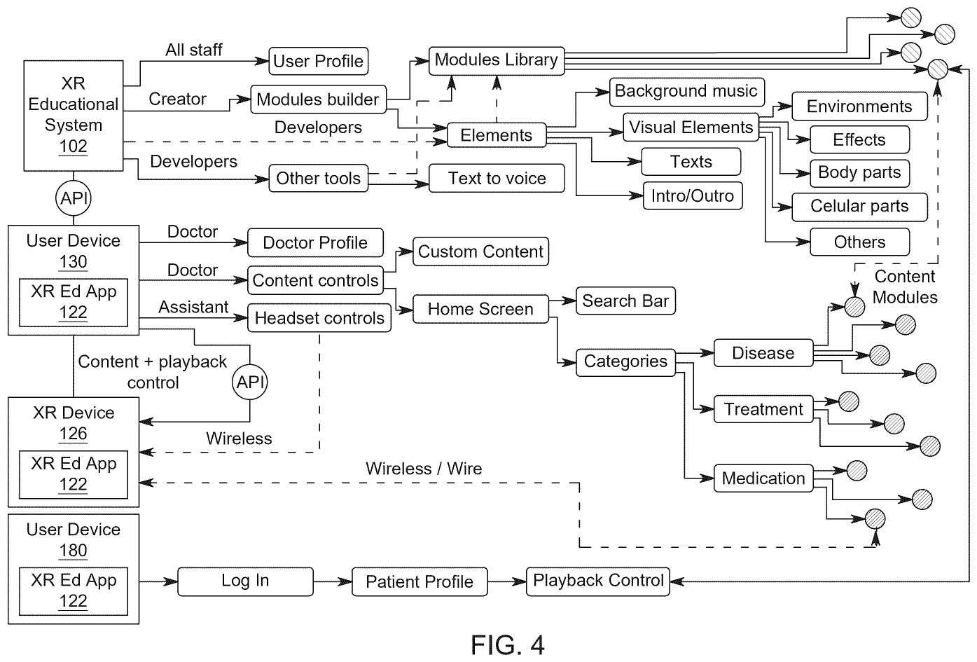

The content builder module 146 operates to create, build, and store content modules; for example, the content module 124. The content module 124 are experiences that deliver patient education on a particular healthcare topic. The content modules 124 can be XR modules that provide an immersive viewing experience. The content modules 124 can also be 2D modules that can be displayed and viewed on various types of user interfaces, such as smartphone display screens. The content builder module 146 can provide an interactive interface where a user can select different elements from the elements library 115 to create the content module 124. Once created, the content module 124 can be stored in the module library 114 for later use or further customization. FIG. 4 shows an example of the processes for generating content modules 124 and providing those content modules 124 to a device to be viewed. In embodiments, the content modules can be created by a user of the XR ED system 102, e.g., staff, developer, etc., and/or a healthcare professional using the XR ED system 102. For example, the healthcare professional 128 can utilize the user device 130 to generate customized modules for patient 118 based on, for instance, the medical records and needs of patient 118.

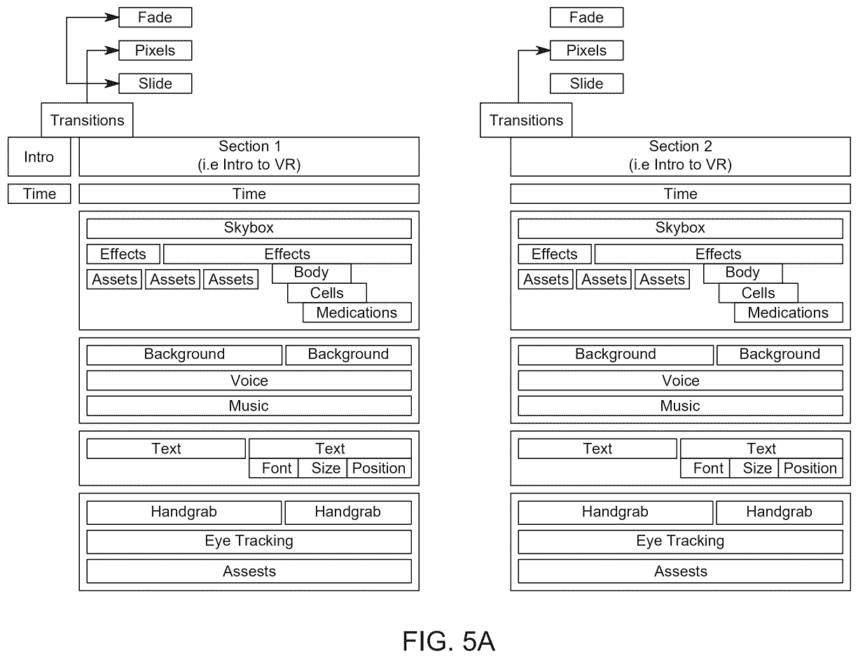

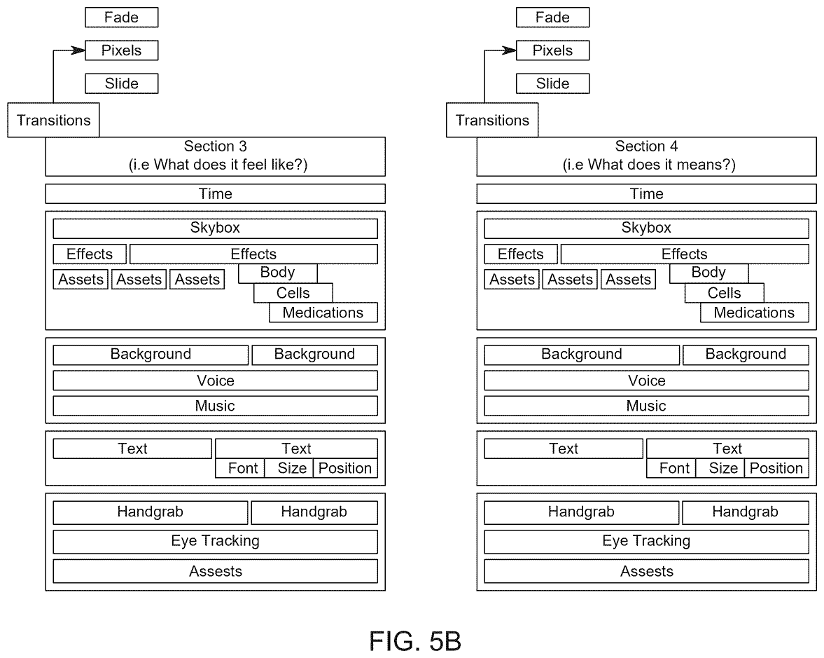

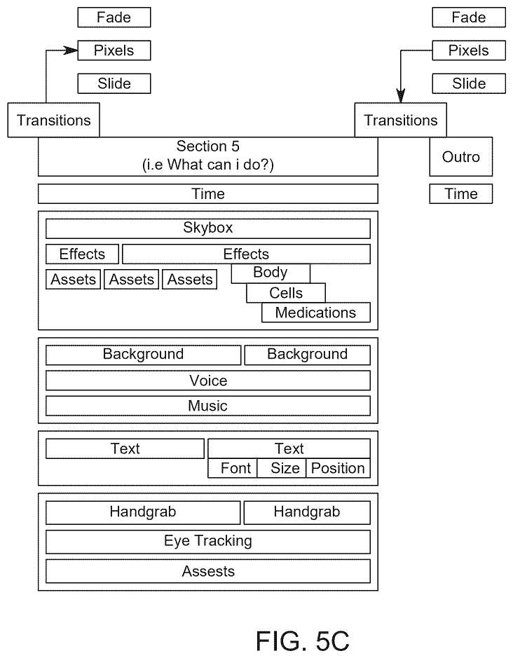

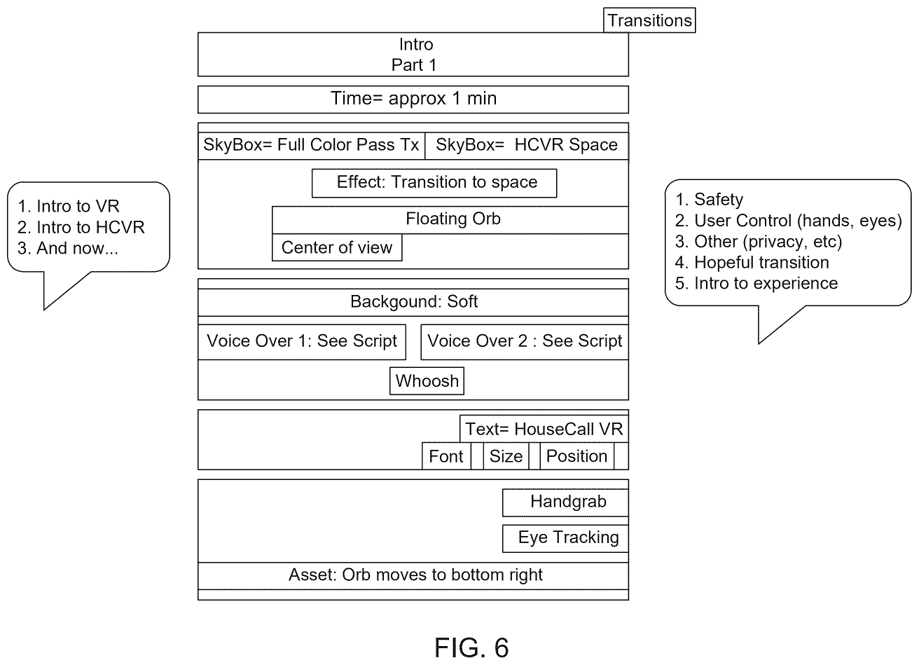

When building an educational experience for diabetes, the content builder module 146 can generate an interface that allows a user, e.g., staff, developer, etc., and/or a healthcare professional to identify and choose each section of the experience (introduction, body, and conclusion). The user can have the opportunity to build each section using prompts, including skybox (environment), sounds, voice, elements/assets, and interaction. FIGS. 5A-5C and 6 illustrate the interface for designing and creating the content modules. As illustrated in FIGS. 5A-5C (one section illustrated in FIG. 6), a content module can be divided into a number of sections that represent location division in the content. The content builder module 146 generates an interface that allows the user to visually select content elements to be placed into the sections, for example, backgrounds, skyboxes, text, music, voice-over, animations, input control, and capture, etc., and align the content elements to design the visual display and temporal flow of the content modules 124. The user can also select transitions between the sections, such as fades, slides, etc.

The profile module 148 operates to establish, track, and enforce access rules based on different user profiles accessing the XR ED system 102 through an interface, e.g., the clinical interface 170 and the patient interface 174, provided by the clinical module 144 and/or the interface module 140. The different profiles can have different access and permission to interact with the XR ED system 102. For example, different profiles can include various users of the XR ED system 102, including creators, developers, patients, doctors, healthcare staff, and the like. All users can be granted access to individual user profiles, facilitating personalized content interaction. For example, developers can be afforded access to other tools and a text to voice, among other features. Creators and developers are provided access to a specialized tool, termed the content builder module 146. The content builder module 146 serves as a gateway to the module library 114 and a diverse set of elements library 115. A unique branching pathway allows creators and developers to transition from the module library 114 directly into the elements library 115.

For healthcare providers, different users can have different profiles for the clinical interface 170; for example, a doctor profile, a doctor plus profile, and an assistant profile. Doctors can have exclusive access to a doctor profile and comfort controls. Comfort controls can encompass custom content and a structured home screen. The home screen features a search bar and categories of disease, treatment, and medication. Each category houses multiple accessible content modules. A doctor plus profile can have access to additional settings. An assistant profile can have access to the VR headset controls, for example, enhancing application interaction. Healthcare providers 128 and patients 118 also have the ability to create profiles for the patient interface 174.

The AI module 150 operates to provide machine learning algorithms and techniques for content creation, content playback, and analysis of feedback. The AI module 150 can communicate with the other modules to provide adaptive and machine learning techniques. Additionally, the AI module 150 can communicate with third-party AI systems to provide an enhanced educational environment. For example, the AI module 150 can include an adaptive learning algorithm that adjusts educational content based on patient interactions and responses. For instance, if a patient struggles with specific concepts, the XR ED system 102 provides additional resources or alternative explanations to enhance understanding. In some embodiments, the AI module 150 can update and reconfigure the content module, in real-time, based on the patient 118 feedback.

The AI module 150 can be used during the creation of the content modules. The AI module 150 can integrate with generative AI algorithms and platforms designed to facilitate the creation of educational content. These AI algorithms and platforms utilize advanced machine learning algorithms to generate personalized educational materials such as images, assets, music, and voice, based on individual needs, preferences, and interactions. The integration with third-party platforms ensures a diverse and engaging learning experience for patients while maintaining efficiency in content creation. The integration with AI algorithms and platforms enables the generation of a wide range of content types, including images, assets, language, music, voice, and other content types. The AI algorithms and platforms analyze patient data and interactions to create content tailored to individual learning styles and needs and adapt in real time based on patient responses. Leveraging the AI algorithms and platforms reduces the time and resources required to create diverse and personalized educational content, allowing for a more streamlined development process.

The AI module 150 can also be used by the healthcare profession 128 and/or the patient 118, using the user device 130, user device 180, and/or the XR device 126, to generate a “future self” of patient 118 for customized content modules 124 for the patient 118 in order to get motivation for better health and following healthcare professional 128 advice. The AI module 150 can generate a generic person representing the patient future self in the content modules 124. The Al module 150 can generate the future self to represent the physical appearance of the patient 118 based on one or more images uploaded into the patient profile. The Al module 150 can manipulate the future self to create a version of the patient within the custom modules that will then be used to interplay with the patient 118 while they are viewing their modules. The AI module 150 can alter the future self to show changes to patient 118 when they take certain actions (e.g., plastic surgery, hip replacement, diet, etc.)

The records module 152 operates to access one or more patient records according to security and privacy regulations, including the Health Insurance Portability and Accountability Act (HIPPA). Ensuring stringent security protocols, the records module 152 can access relevant patient medical records, refining the educational content to match individual health profiles. With appropriate permissions, the XR ED system 102 can utilize patient data to customize the VR experience, aligning with individual medical histories, learning styles, and preferences. In some embodiments, the records module 152 can utilize one or more block chain schemes to secure the data stored for the patients.

In embodiments, while the output of the XR ED system 102 can be educational content, the XR ED system 102 can also produce detailed user analytics data and learning progress reports. These by-products can be valuable resources for healthcare professionals, educators, researchers and others, offering insights into user behavior, user preferences, learning patterns, and the effectiveness of different educational strategies. Additionally, the XR ED system 102 adaptability means it could be configured to create personalized learning modules or training programs, serving as a versatile tool in various educational contexts outside of healthcare.

In embodiments, the XR ED system 102 can include:
User Analytics: Incorporating advanced user analytics could provide deeper insights and data into user behavior and learning patterns, enabling further personalization and optimization of content.
Community Features: Adding community forums or discussion groups within the software could foster peer-to-peer learning and support, enhancing the overall educational experience.
Content Expansion: Continuously updating and expanding the library of educational content to cover a broader range of healthcare topics and disciplines would enhance the software's utility and relevance.
Accessibility Enhancements: Further developments in accessibility features could make the software more inclusive and usable by a wider range of individuals with varying needs.

In embodiments, a process of using the XR ED system 102 can include Stage 1 Login: Healthcare professionals log in to the clinical interface 170 to access and manage educational content. Stage 2 Select Content: Professionals choose relevant educational experiences tailored to individual patient needs. Stage 3 Send to Patient: The selected content is securely sent to the patient's device/account. Stage 4 Access & Interact: Patients access the content via VR headsets, web browsers, or smartphone apps, interacting with personalized, adaptive learning modules. Stage 5 Receive Feedback: Patients receive immediate feedback through quizzes and interactive scenarios, enhancing learning. Stage 6 Track Progress: Both patients and healthcare professionals can monitor progress and adapt content accordingly. Stage 7 Utilize Additional Features: Users can opt to engage with optional features like gamification, multi-sensory modules, and community forums for an enriched experience.

Besides delivering educational content, the XR ED system 102 can generate valuable user analytics and learning progress reports. These data can be instrumental in health analytics, contributing to healthier lifestyles and enabling hospitals and health systems to realize cost savings through enhanced efficiencies. The insights derived can inform personalized healthcare strategies, optimize patient education, and improve overall health outcomes. Additionally, the adaptability of the software allows for the creation of tailored learning modules, making it a versatile tool across diverse educational and healthcare settings.

In embodiments, the XR ED system 102 can also generate:
1. Educational Content: Generates personalized educational experiences for patients, enhancing their understanding of health conditions and treatments.
2. User Analytics: Produces valuable data on user interactions, learning progress, and emotional responses, contributing to personalized healthcare strategies.
3. Health Insights: Offers insights derived from user data to inform healthcare practices, optimize patient education, and improve health outcomes.
4. Adaptive Learning Modules: Creates tailored learning modules adaptable across diverse educational and healthcare settings.
5. Efficiency Reports: Provides reports on learning efficacy and user engagement, aiding in the optimization of educational content and delivery methods.

These components and modules form a comprehensive and interactive extended reality patient education software system. The invention leverages advanced technologies such as XR, AI, and adaptive learning algorithms to deliver standardized, general, adaptive, interactive, and personalized educational experiences covering all areas of the healthcare spectrum. The synergy of these elements results in a revolutionary approach to patient education, addressing the limitations of existing methods and significantly enhancing patient understanding and retention.

Returning to FIG. 1B, the processing device 104, the communication device 106, and the memory device 108 can be interconnected via a system bus. The system bus can be and/or include a control bus, a data bus, an address bus, and the like. The processing device 104 can be and/or include a processor, a microprocessor, a computer processing unit (“CPU”), a graphics processing unit (“GPU”), a neural processing unit, a physics processing unit, a digital signal processor, an image signal processor, a synergistic processing element, a field-programmable gate array (“FPGA”), a sound chip, a multi-core processor, and the like. As used herein, “processor,” “processing component,” “processing device,” and/or “processing unit” can be used generically to refer to any or all of the aforementioned specific devices, elements, and/or features of the processing device. While FIG. 1 illustrates a single processing device 104, the XR ED system 102 can include multiple processing devices 104, whether the same type or different types.

The memory device 108 can be and/or include one or more computerized storage media capable of storing electronic data temporarily, semi-permanently, or permanently. The memory device 108 can be or include a computer processing unit register, a cache memory, a magnetic disk, an optical disk, a solid-state drive, and the like. The memory device can be and/or include random access memory (“RAM”), read-only memory (“ROM”), static RAM, dynamic RAM, masked ROM, programmable ROM, erasable and programmable ROM, electrically erasable and programmable ROM, and so forth. As used herein, “memory,” “memory component,” “memory device,” and/or “memory unit” can be used generically to refer to any or all of the aforementioned specific devices, elements, and/or features of the memory device 108. While FIG. 1 illustrates a single memory device 108, the XR ED system 102 can include multiple memory devices 108, whether the same type or different types.

The communication device 106 enables the XR ED system 102 to communicate with other devices and systems. The communication device 106 can include hardware and/or software for generating and communicating signals over a direct and/or indirect network communication link. As used herein, a direct link can include a link between two devices where information is communicated from one device to the other without passing through an intermediary. For example, the direct link can include a Bluetooth™ connection, a Zigbee connection, a Wifi Direct™ connection, a near-field communications (“NFC”) connection, an infrared connection, a wired universal serial bus (“USB”) connection, an ethernet cable connection, a fiber-optic connection, a firewire connection, a microwire connection, and so forth. In another example, the direct link can include a cable on a bus network programming installed on a processor, such as the processing component, coupled to the antenna.

An indirect link can include a link between two or more devices where data can pass through an intermediary, such as a router, before being received by an intended recipient of the data. For example, the indirect link can include a WiFi connection where data is passed through a WiFi router, a cellular network connection where data is passed through a cellular network router, a wired network connection where devices are interconnected through hubs and/or routers, and so forth. The cellular network connection can be implemented according to one or more cellular network standards, including the global system for mobile communications (“GSM”) standard, a code division multiple access (“CDMA”) standard such as the universal mobile telecommunications standard, an orthogonal frequency division multiple access (“OFDMA”) standard such as the long term evolution (“LTE”) standard, and so forth.

The XR ED system 102 can communicate with one or more network resources via the network 116. The one or more network resources can include external databases, social media platforms, search engines, file servers, web servers, or any type of computerized resource that can communicate with the XR ED system 102 via the network 116.

In embodiments, the components and functionality of the XR ED system 102 can be hosted and/or instantiated on a “cloud” and/or “cloud service.” As used herein, a “cloud” and/or “cloud service” can include a collection of computer resources that can be invoked to instantiate a virtual machine, application instance, process, data storage, or other resources for a limited or defined duration. The collection of resources supporting a cloud can include a set of computer hardware and software configured to deliver computing components needed to instantiate a virtual machine, application instance, process, data storage, or other resources. For example, one group of computer hardware and software can host and serve an operating system or components thereof to deliver to and instantiate a virtual machine. Another group of computer hardware and software can accept requests to host computing cycles or processor time, to supply a defined level of processing power for a virtual machine. A further group of computer hardware and software can host and serve applications to load on an instantiation of a virtual machine, such as an email client, a browser application, a messaging application, or other applications or software. Other types of computer hardware and software are possible.

In embodiments, the components and functionality of the XR ED system 102 can be and/or include a “server” device. The term server can refer to functionality of a device and/or an application operating on a device. The server device can include a physical server, a virtual server, and/or cloud server. For example, the server device can include one or more bare-metal servers such as single-tenant servers or multiple-tenant servers. In another example, the server device can include a bare metal server partitioned into two or more virtual servers. The virtual servers can include separate operating systems and/or applications from each other. In yet another example, the server device can include a virtual server distributed on a cluster of networked physical servers. The virtual servers can include an operating system and/or one or more applications installed on the virtual server and distributed across the cluster of networked physical servers. In yet another example, the server device can include more than one virtual server distributed across a cluster of networked physical servers.

Various aspects of the systems described herein can be referred to as “content” and/or “data.” Content and/or data can be used to refer generically to modes of storing and/or conveying information. Accordingly, data can refer to textual entries in a table of a database. Content and/or data can refer to alphanumeric characters stored in a database. Content and/or data can refer to machine-readable code. Content and/or data can refer to images. Content and/or data can refer to audio and/or video. Content and/or data can refer to, more broadly, a sequence of one or more symbols. The symbols can be binary. Content and/or data can refer to a machine state that is computer-readable. Content and/or data can refer to human-readable text.

Various of the devices in the interactive health care environment 100 can include a user interface for outputting information in a format perceptible by a user and receiving input from the user. The user interface can display graphical user interfaces (“GUIs”) generated by the XR ED system 102 and/or the XR Ed application 122. The user interface can include a display screen such as a light-emitting diode (“LED”) display, an organic LED (“OLED”) display, an active-matrix OLED (“AMOLED”) display, a liquid crystal display (“LCD”), a thin-film transistor (“TFT”) LCD, a plasma display, a quantum dot (“QLED”) display, and so forth. The user interface can include an acoustic element such as a speaker, a microphone, and so forth. The user interface can include a button, a switch, a keyboard, a touch-sensitive surface, a touchscreen, a camera, a fingerprint scanner, and so forth. The touchscreen can include a resistive touchscreen, a capacitive touchscreen, and so forth.

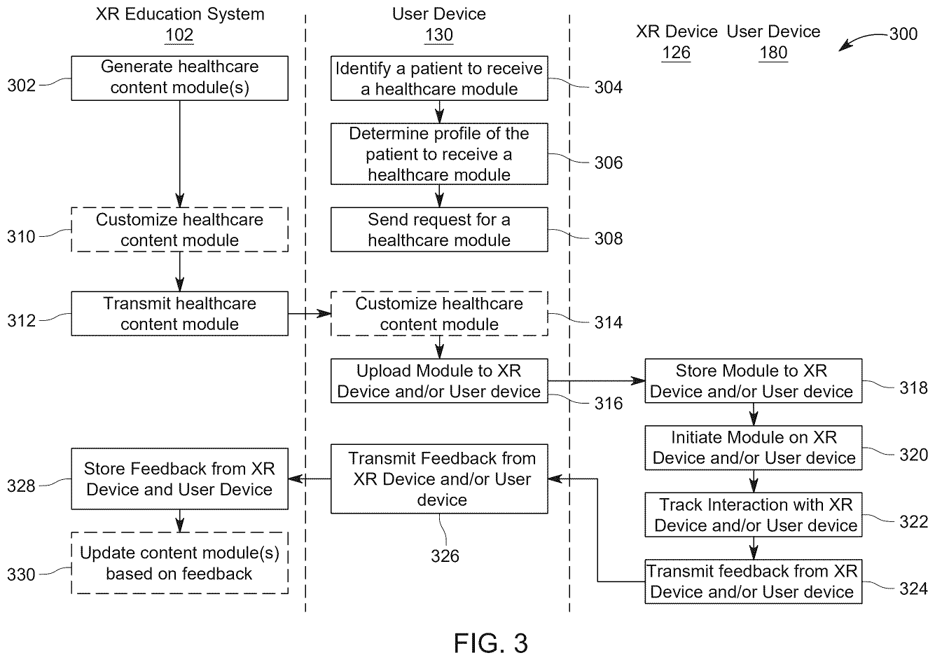

FIG. 3 illustrates a method 300 for creating and delivering content modules, according to aspects of the present disclosure. While FIG. 3 illustrates examples of stages of the method 300, additional stages can be added, and existing stages can be reordered, removed, and/or modified.

As illustrated, the method 300 can begin at stage 302. In stage 302, health care content modules can be created. While stage 302 is illustrated as occurring first, the creation of the content modules can occur at any time during the method 300. For example, the content modules 124 can be created using the process illustrated in FIG. 4 and the interfaces illustrated in FIGS. 5A-5C and 6.

In stage 304, a patient can be identified to receive a content module. For example, referring to FIGS. 1A and 1B, a patient 118 can visit a healthcare provider location 128. The healthcare professional 128 can determine that the patient may benefit for viewing a content module 124.

In stage 306, a profile of the patient to receive the healthcare module can be determined. For example, the healthcare professional 128, using the user device 130, can access the healthcare records for the patient 118. The profile of the patient can include relevant health and personal information including diagnoses. The profile can also include healthcare content modules that have been viewed by the patient 118.

In stage 308, a request for a healthcare module 308 is sent. For example, the healthcare professional 128, using the user device 130, can request a content module 124 from the XR ED system 102. In some embodiments, the request can include a request for a specific type of content module based on the patient profile. For instance, the patient 118 can receive a diagnosis of diabetes, and the healthcare professional 128 may desire to educate the patient 118 on the details and care for diabetes.

In some embodiments, the healthcare professional 128, using the user device 130, can request a custom content module. In this example, the healthcare professional 128, using the user device 130, can provide details of the patient 118 so that a custom content module can be created for the patient 118.

In stage 310, once the request is received, a healthcare content module can be optionally customized. For example, the healthcare professional 128, using the user device 130, can provide details of the patient 118 so that a custom content module can be created for the patient 118. For instance, patient 118 can receive a diagnosis of diabetes, and the healthcare professional 128 may desire to educate the patient 118 on the details and care for diabetes. To customize the module, the healthcare professional 128, using the user device 130, can provide other preexisting conditions of the patient in order to tailor a content module for the patient 118.

In stage 312, the healthcare content module is transmitted. The content module can be a customized module or a standard content module. For example, the XR ED system 102 can transmit the content module 124 to the user device 130. In another example, the XR ED system 102 can transmit the content module 124 directly to the XR device 126.

Once received, in stage 314 the healthcare content module can be optionally customized. For example, when the content module 124 is received at the user device, the healthcare professional 128 can customize the content module 124 to the patient 118 using the XR Ed app 122 on user device 130.

Once ready, in stage 316, the healthcare content module can be uploaded to the XR device and/or other user device. For example, the healthcare professional 128, using the user device 130, can upload the content module 124 to the XR device 126. Once received, in stage 318, the XR device and/or other user device can store the content module.

In stage 320, the content module can be initiated on the XR device and/or other user device. In stage 322, a user of the XR device and/or other user device can view the content module and interact with the XR device and/or other user device. In stage 322, the XR device and/or other user device can track the user interaction.

In stage 324, the XR device and/or other user device can transmit feedback of the user interaction. In some embodiments, the XR device 126 and/or user device 180 can transmit the feedback to the user device 130. In some embodiments, the XR device 126 and/or user device 180 can transmit the feedback directly to the XR ED system 102.

In stage 328, the feedback from the XR device and/or from the user device can be stored. For example, the XR ED system 102 can store the feedback 136 from the user device 130 and/or the XR device 126 for viewing the content module 124. The feedback 136 can include feedback from the XR device 126 such as user input as the content module 124 is viewed and/or biometric data tracked by the XR device 126 as the user views the content module 124. The feedback 136 can also include feedback from user device 180 as the user views the content module 124 and comments and input from the healthcare professional 128. In stage 330, the content modules can be optionally updated based on the feedback.

In embodiments, the feedback from the XR device and/or other user device can be utilized to update the content module 124, in real-time, as the content module is being viewed. For example, in a module on hypertension, the user can actively choose the next section of the module by deciding to either learn about sodium reduction or exercise as mititgating factors (“choose your own adventure”). In another example, the module could be automatically slowed down if the user's biometric data feedback from the XR device and/or other user device indicates stress.

As used in the description herein and throughout the claims that follow, “a”, “an”, and “the” include plural references unless the context clearly dictates otherwise. Also, as used in the description herein and throughout the claims that follow, the meaning of “in” includes “in” and “on” unless the context clearly dictates otherwise. While the above is a complete description of specific examples of the disclosure, additional examples are also possible. Thus, the above description should not be taken as limiting the scope of the disclosure which is defined by the appended claims along with their full scope of equivalents.

The foregoing disclosure encompasses multiple distinct examples with independent utility. While these examples have been disclosed in a particular form, the specific examples disclosed and illustrated above are not to be considered in a limiting sense as numerous variations are possible. The subject matter disclosed herein includes novel and non-obvious combinations and sub-combinations of the various elements, features, functions and/or properties disclosed above both explicitly and inherently. Where the disclosure or subsequently filed claims recite “a” element, “a first” element, or any such equivalent term, the disclosure or claims is to be understood to incorporate one or more such elements, neither requiring nor excluding two or more of such elements. As used herein regarding a list, “and” forms a group inclusive of all the listed elements. For example, an example described as including A, B, C, and D is an example that includes A, includes B, includes C, and also includes D. As used herein regarding a list, “or” forms a list of elements, any of which may be included. For example, an example described as including A, B, C, or D is an example that includes any of the elements A, B, C, and D. Unless otherwise stated, an example including a list of alternatively-inclusive elements does not preclude other examples that include various combinations of some or all of the alternatively-inclusive elements. An example described using a list of alternatively-inclusive elements includes at least one element of the listed elements. However, an example described using a list of alternatively-inclusive elements does not preclude another example that includes all of the listed elements. And, an example described using a list of alternatively-inclusive elements does not preclude another example that includes a combination of some of the listed elements. As used herein regarding a list, “and/or” forms a list of elements inclusive alone or in any combination. For example, an example described as including A, B, C, and/or D is an example that may include: A alone; A and B; A, B and C; A, B, C, and D; and so forth. The bounds of an “and/or” list are defined by the complete set of combinations and permutations for the list.

It should be understood, of course, that the foregoing relates to exemplary embodiments of the disclosure and that modifications can be made without departing from the spirit and scope of the disclosure as set forth in the following claims.

What is claimed is:

1. A method of delivering interactive content, comprising:
determining a content module to be played on a user device;
downloading the content module to the user device;
executing the content module on the user device; and
tracking a user's progress on viewing the content module on the user device.

2. The method of claim 1, the method further comprising:
compiling feedback collected based on user interaction with the content module executing the user device.

3. The method of claim 1, the method further comprising:
customizing the content module based on a medical history of an intended user of the content module.

4. The method of claim 1, wherein determining the content module comprises:
generating a visual representation of an intended user of the content module based on one or more images of the intended user; and
integrating the visual representation of the intended user into the content module.

5. The method of claim 4, wherein determining the content module comprises:
modifying the visual representation of the intended user based on a progress of one or more health conditions of the intended user.

6. A method of delivering interactive content, comprising:
storing content elements in an elements library;
receiving a request for a content module, the request comprising an identification of a subject of the content module;
extracting, from the elements library, one or more content elements based on the subject of the content module;
generating the content module from the one or more content elements; and
storing the content module in a computer-readable storage medium.

7. The method of claim 6, further comprising:
providing the content module to an extended reality playback device or other user device operated by a user; and
receiving, from the extended reality playback device or other user device, feedback collected based on user interaction with the content module executing on the extended reality playback device or other user device.

8. The method of claim 7, wherein the feedback comprises at least one of biometric feedback collected by the extended reality playback device or other user device, user input during the user interaction with the content module executing on the extended reality playback device or other user device, and third-party feedback based on the user interaction with the content module executing on the extended reality playback device or other user device.

9. The method of claim 8, wherein the content module is a healthcare content module, the user is a patient, and the third-party observer is a healthcare professional.

10. The method of claim 7, further comprising:
modifying the content module based on the feedback collected based on user interaction with the content module executing on the extended reality playback device or other user device.

11. The method of claim 10, wherein the modifying of the content module based on the feedback comprises modifying the content module in real-time while the content module executes on the extended reality playback device or other user device.

12. The method of claim 10, wherein the modifying of the content module is performed by one or more machine learning algorithms trained to modify the content module based on the feedback.

13. The method of claim 6, wherein the content module is generated by one or more machine learning algorithms trained to generate the content module based on one or more inputs comprising the subject of the content module.

14. A system of delivering interactive content, comprising:
a content generation system configured to:
store content elements in an elements library,
receive a request for a content module, the request comprising an identification of a subject of the content module,
extract, from the elements library, one or more content elements based on the subject of the content module,
generate the content module from the one or more content elements, and
storing the content module in a computer-readable storage medium; and
an extended reality content playback device or other user device coupled to the content generation system by one or more networks and configured to:
receive the content module, and
execute the content module for viewing and interaction by a user.

15. The system of claim 14, the system further comprising:
a user device coupled to the content generation system and the extended reality content playback device or other user device by the one or more networks and configured to:
configure the content module for playback on the extended reality content playback device.

16. The system of claim 15, wherein the extended reality playback device or other user device is further configured to:
collect feedback based on user interaction with the content module executing on the extended reality playback device or other user device, and
provide the feedback to the content generation system.

17. The system of claim 16, wherein the content generation system is further configured to:
modify the content module based on the feedback received.
